(19) United States
(12) Patent Application Publication
(10) Pub. No.: US 2016/0317691 A1
(43) Pub. Date: Nov. 3, 2016

(54) VEHICULAR AIR FRESHENING SYSTEM

(71) Applicant: Cara Sorto, Webster, FL (US)

(72) Inventor: Cara Sorto, Webster, FL (US)

(21) Appl. No.: 14/699,355

(22) Filed: Apr. 29, 2015

(51) Int. Cl. A61L 9/14; B60R 7/08

(52) U.S. Cl. CPC A61L 9/14; B60R 7/08

(57) ABSTRACT

A vehicular air freshening system includes a reservoir containing an odor suppressing agent. The reservoir is connected to a dispensing unit via a supply line. A controller is located within the dispensing unit and communicates with a remote operation unit.

### TECHNICAL FIELD

The present invention relates generally to air fresheners, and more particularly to an air freshening system for use within a vehicle.

### BACKGROUND

The statements in this section merely provide background information related to the present disclosure and may not constitute prior art.

Operators of modern automobiles, trucks and other vehicles tend to demand a more comfortable environment in those vehicles than was expected in the past. Today, motor vehicle operators expect more from a vehicle than mere transportation. Accordingly, manufacturers have generally provided heating, ventilating and air conditioning systems which allow a vehicle driver to shut out the outside environment during the majority of time spent in the vehicle. These features combined with improved construction techniques result in vehicle interiors that are effectively sealed off from the outside environment.

Despite improvements in design and materials, all vehicles will eventually absorb or acquire unpleasant odors in the course of their use. Whether it is the result of smoking in the interior of the vehicle, spilled food or drink, and/or poor interior cleanliness, the gradual buildup of unpleasant odors is a virtual certainty.

There are many known devices which have been previously developed in an attempt to combat this situation. For example, there are many known scented hang tags which are coated with a scent and suspended from the vehicle rear view mirror. Additionally, there are known clip devices which are also coated with a scent and positioned against the vehicle air conditioning vent. Although useful for creating a short term solution, each of these devices suffer from several drawbacks in the fact that they block useable space, and their effectiveness dissipates over a period of days. More specifically, each of these devices tends to produce an overabundance of scent when first deployed, and after a few days they do not produce enough scent.

Accordingly, it would be beneficial to provide a vehicular air freshening system that is unobtrusive when installed, does not affect the air flow of the vehicular ventilation system, and which can repeatedly discharge measured amounts of air freshening solution.

### SUMMARY OF THE INVENTION

The present invention is directed to a vehicular air freshening system. One embodiment of the present invention can include a reservoir for storing an odor suppressing agent. The reservoir can be connected to a dispensing unit via a supply line for dissemination into the vehicle passenger compartment. A controller can be positioned within the dispensing unit and can receive a user instruction from a remote operation unit.

Another embodiment of the present invention can include a timer module which can function to automatically activate the dispensing unit at a predetermined time.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a perspective view of the vehicular air freshening system that is useful for understanding the inventive concepts disclosed herein.

FIG. 2 is a cutout view of the dispensing unit of the vehicular air freshening system, in accordance with one embodiment of the invention.

FIG. 3 is a simplified block diagram of the controller of the vehicular air freshening system, in accordance with one embodiment of the invention.

FIG. 4 is a perspective view of the vehicular air freshening system in operation, in accordance with one embodiment of the invention.

FIG. 5 is a front view of the vehicular air freshening system, in accordance with another embodiment of the invention.

FIG. 6 is a front view of the vehicular air freshening system, in accordance with another embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

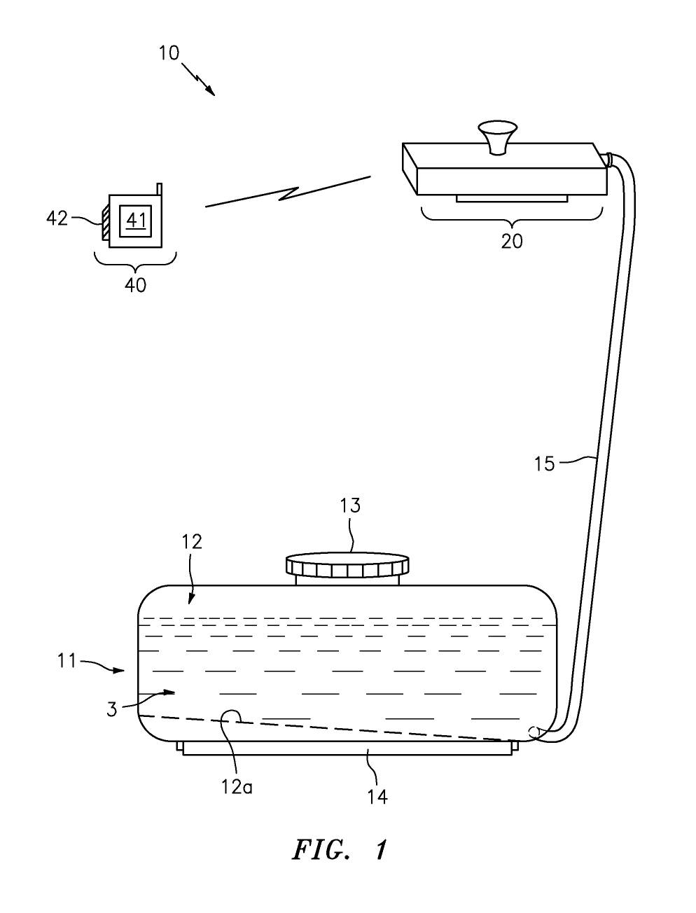

Identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1.

Although illustrated with respect to an aftermarket component and including connectors for the same, the invention is not to be construed as limiting in this regard. As such, it is contemplated that the device can be incorporated into the design and construction of new vehicles an OEM device, wherein placement components may be different than what is illustrated.

FIGS. 1-4 illustrate one embodiment of a vehicular air freshening system 10 that is useful for understanding the inventive concepts disclosed herein. As shown, the system can include a liquid reservoir 11, a dispensing unit 20, a controller 30 and a remote operation unit 40.

The reservoir 11 can function to receive and store any type of agent that is used for suppressing and/or eliminating odors. In one embodiment, the agent can include a liquid solution however other agent compositions are also contemplated. In this regard, the reservoir can include a main body having a plurality of sides that define a watertight space 12 for storing the agent 3. As shown, the bottom portion of the watertight space 12a can preferably be angled so as to direct the deodorizing agent towards a supply line 15 for transport to the dispensing unit described below. A removable cap 13 or other such port can be provided along the reservoir body so as to allow the agent to be refilled as needed.

In the preferred embodiment, the reservoir can be constructed from injection molded plastic; however, any number of different materials that are suitable for long term exposure to heat and vibrations caused by the movement of the vehicle are also contemplated.

In one embodiment, a first connector 14 can be provided in order to allow a user to mount the reservoir to the vehicle, and preferably within the dashboard area of an automobile. As described herein, a “connector” can include any number of different elements capable of securing two items together in either a permanent or a nonpermanent manner. Several nonlimiting examples include opposing strips of hook and loop material (i.e. Velcro®), magnetic elements, tethers such as straps and zip ties, as well as compression fittings such as screws, nuts, bolts and other such hardware.

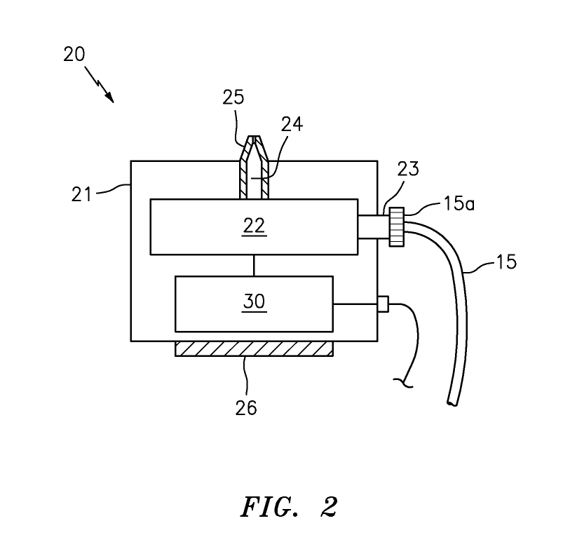

The dispensing unit 20 can function to receive the deodorizing agent from the reservoir, and to distribute the same into the vehicle compartment. As shown best in FIG. 2, the dispensing unit can include a main body 21 for housing a spray pump 22, a pump intake connector 23, a spray line 24, a spray nozzle 25, and a controller 30.

The main body 21 can include any shape and can be constructed from any number of different materials such as plastic, for example. A second connector 26 can preferably be included to facilitate mounting the dispenser onto a vehicle surface. In the preferred embodiment, the second connector 26 can include double sided adhesive tape, that is positioned along the bottom surface of the main body 21, in order to allow the dispenser to be mounted onto an automobile dashboard at a location adjacent to the windshield. As will be described below, such placement allows the most effective dissemination of the atomized solution. Of course, any number of other connectors and locations are also contemplated.

The spray pump 22 can include any electrically operated gas/fluid pump capable of receiving the deodorizing product from the reservoir/supply line 15 and disseminating the same through the spray line 24 and nozzle 25. Although the supply line can be permanently affixed to the pump intake, in the preferred embodiment, the supply line and intake can include complementary valve fasteners 15a, such as threaded valve connectors, for example, so as to allow the two devices to be secured together in a removable manner.

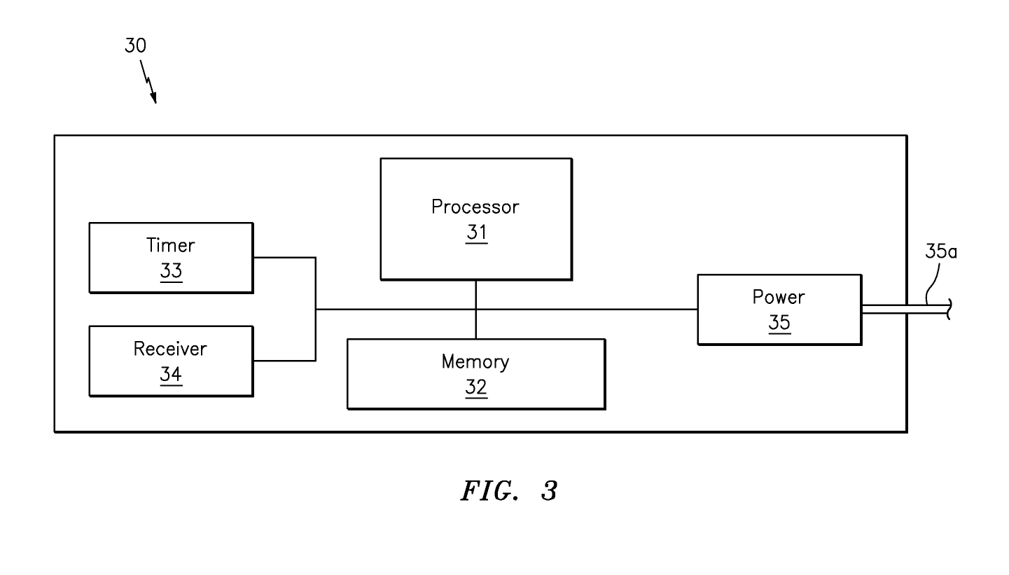

The controller 30 can function to control an operation of the dispensing unit. FIG. 3 illustrates one embodiment of a controller that includes a processor 31 that is conventionally connected to an internal memory 32, a timer module 33, a receiver 34, and a power module 35.

Although illustrated as separate elements, those of skill in the art will recognize that one or more system components may be, or include one or more printed circuit boards (PCB) containing an integrated circuit or circuits for completing the activities described herein. The CPU may be one or more integrated circuits having firmware for causing the circuitry to complete the activities described herein.

The processor/CPU 31 can act to execute program code stored in the memory 32 in order to allow the device to perform the functionality described herein. Likewise, a timer module 33 can be provided, and can function to accurately measure the passage of time. As described herein, the timer module can be provided as a function of the processor or can include a separate physical circuit. In either instance, processors and timers are extremely well known in the art, therefore no further description will be provided.

Memory 32 can act to store operating instructions in the form of program code for the processor 31 to execute. As described herein, memory can include one or more physical memory devices such as, for example, local memory and/or one or more bulk storage devices. Additionally, memory can also include one or more cache memories that provide temporary storage of at least some program code in order to reduce the number of times program code must be retrieved from the bulk storage device during execution. Each of these devices are well known in the art.

The receiver 34 can act to receive a signal from an outside device, and more particularly the below described remote operation unit 40. In one preferred embodiment, the receiver can include a variable radio wave receiver having a unique radio frequency chip capable of receiving and translating a plurality of independent radio frequencies which can be sent to the processor 31 and/or the memory 32.

Although described above as using radio transmission, reception and frequencies, other communication mediums and their associated components are also contemplated. For example, infrared (IR), Bluetooth, RFID, microwave and other known communication mediums can also be utilized without deviating from the scope and spirit of the inventive concepts disclosed herein.

The power module 35 can function to receive, convert and/or distribute electrical power to the device components. In the preferred embodiment, the power module can be connected to the vehicles 12 volt electrical system via a cable 35a. Of course, the power module can include any number of other components capable of receiving any type of electrical power (A/C or D/C) and converting the same for use by the device components.

The remote operation unit 40 can include a user button 41 for transmitting a signal to the controller to activate the spray pump. In the preferred embodiment, the unit can be battery operated, and can include a third connector 42 such as double sided tape, for example. In this regard, the user can position the switch at any desirable location within the vehicle, and is not limited to the dash board. Of course, other embodiments are also contemplated wherein the receiver and the remote operation unit are physically connected via an electrical wire, for example, in order to control the operation of the device.

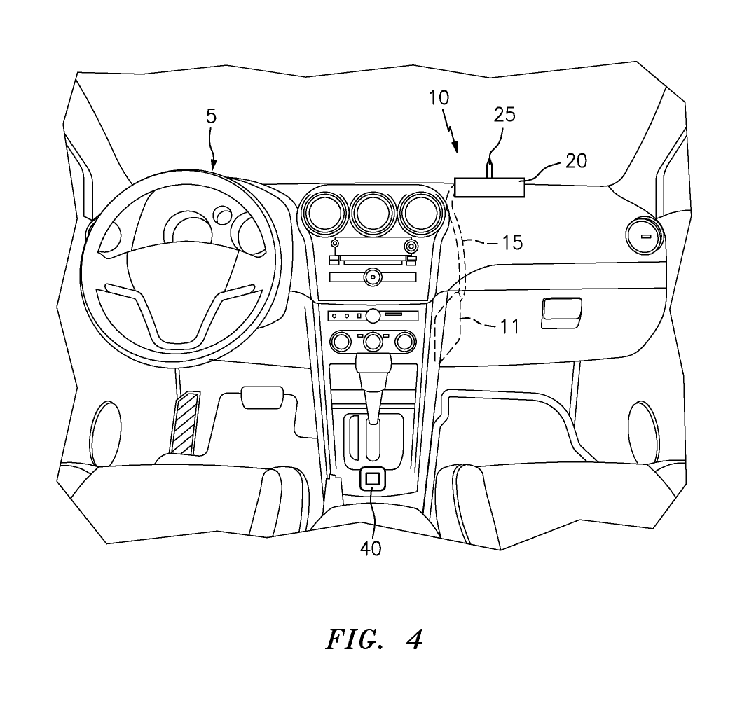

FIG. 4 illustrates one embodiment of the system 10 installed into a vehicle 5. As shown, the reservoir 11 can be positioned at any number of different locations such as behind/under the dashboard, the vehicle trunk or within the engine compartment, for example. The dispensing unit can be placed anywhere within the vehicle compartment and preferably at a location that is adjacent to either the front or rear windshield, so as to allow the airflow from the vehicle environmental system to distribute the same throughout the interior. However, it is also possible to mount the dispensing unit into the roof of the vehicle, or other such locations when included as an OEM component. Finally, the remote operation unit 40 can be positioned at any convenient location, as determined by the user.

In operation, the spray pump 22 is actuated by the receipt of a signal by the user button 41 which instructs the timer and processor to energize the spray pump for a predetermined period of time. In one embodiment, the actuation can persist for a period of 1-2 seconds, however other times are contemplated. Once activated, the spray pump 22 draws up the stored deodorizing agent 3 from the supply line 15 and reservoir 11, and ejects it out through the spray nozzle 25 where it is atomized.

In addition to the above, the system will preferably be connected to the vehicle electric system so as to receive power when the vehicle is in operation (e.g., most automobile accessories only function when the ignition is turned on). In this regard, the memory can store operating instructions wherein the spray pump can be automatically activated at a second period of time, and without receiving an instruction from the remote operation unit. For example, each time the system is energized with power from the vehicle, the timer can count down 30 minutes, or any other amount of time. Upon reaching zero, the timer can notify the processor, which can immediately trigger the spray pump 22. Such a feature can allow the system to continuously deodorize the vehicle, thereby preventing accumulation of odors over time.

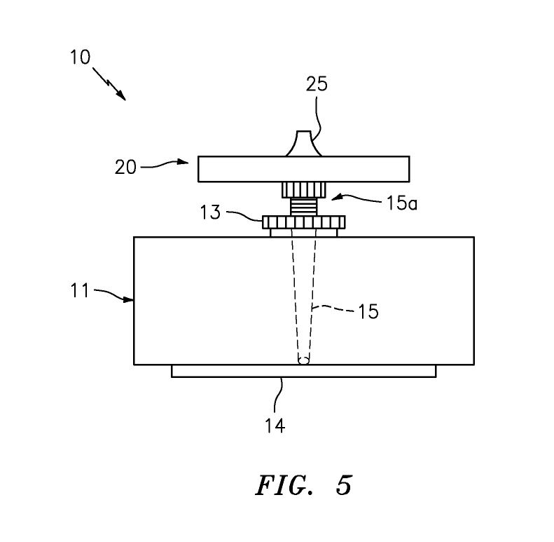

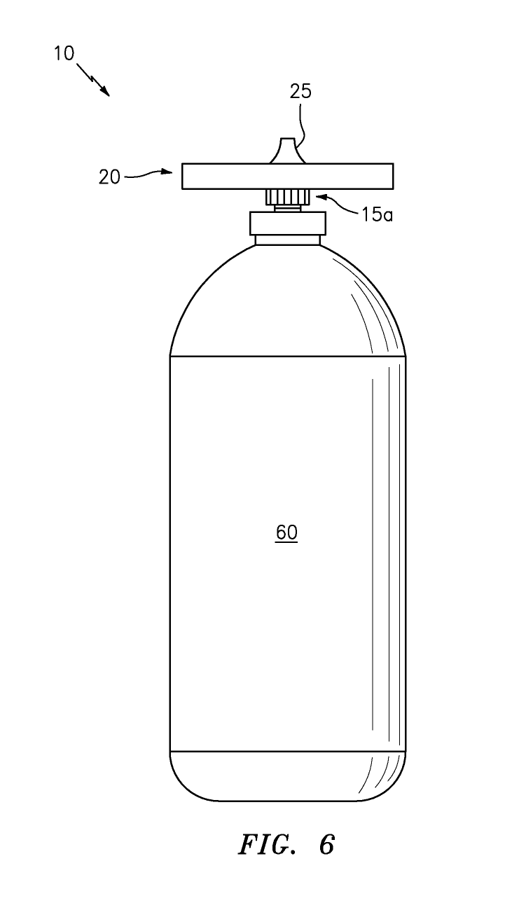

Although described above with respect to a reservoir that is connected to the dispensing unit via an elongated supply line, other embodiments are also contemplated. For example, FIGS. 5 and 6 illustrate another embodiment of the system 10 wherein the reservoir 11 is directly connected to the dispensing unit via the valve connector 15a. As shown, in this configuration, the valve connector can preferably be integrated into the removable cap 13, so as to allow the reservoir to be refilled when needed. Such a feature provides a more compact manner of achieving the inventive concepts. Finally, it is also contemplated that the reservoir itself can be replaced with an aerosol canister 60 containing a pressurized solution. The canister 60 can also be connected to the spray pump via a valve fastener 15a, or other known device, and the pump unit 23 can receive the pressurized solution and distribute the same, as described above.

As described herein, one or more elements of the system 10 can be secured together utilizing any number of known attachment means such as, for example, screws, glue, compression fittings and welds, among others. Moreover, although the above embodiments have been described as including separate individual elements, the inventive concepts disclosed herein are not so limiting. To this end, one of skill in the art will recognize that one or more individual elements may be formed together as one continuous element, either through manufacturing processes, such as welding, casting, or molding, or through the use of a singular piece of material milled or machined with the aforementioned components forming identifiable sections thereof.

As to a further description of the manner and use of the present invention, the same should be apparent from the above description. Accordingly, no further discussion relating to the manner of usage and operation will be provided.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A vehicular air freshening system for distributing an odor suppressing agent, said system comprising:
a remote operation unit for sending a signal upon receiving a user input;
a reservoir for storing an odor suppressing agent;
a supply line that is connected to the reservoir; and
a dispensing unit that includes
a main body housing an electric spray pump that is connected to the supply line,
wherein said dispensing unit functions to receive and dispense the odor suppressing agent upon receipt of the signal.

2. The system of claim 3, further comprising:
a controller that is positioned within the main body of the dispensing unit, said controller comprising:
a receiver that functions to receive the signal from the remote operation unit;
a timer module for determining a passage of time;
a power module that functions to power the dispensing unit;
a memory for storing operating instructions; and
a processor that is in communication with each of the memory, power module, timer module and receiver.

3. The system of claim 2, wherein the signal consists of a wireless signal.

4. The system of claim 2, wherein the memory is encoded with instructions for instructing the processor to automatically activate the spray pump upon receiving a signal from the timer that a predetermined period of time has elapsed.

5. The system of claim 1, wherein the dispensing unit further comprises:
a pump intake connector that is in communication with the supply line and the spray pump;
a spray line that is in communication with the spray pump; and
a spray nozzle that is in communication with the spray line, said spray nozzle functioning to atomize the odor suppressing agent.

6. The system of claim 1, wherein the odor suppressing agent is a liquid, and the reservoir includes a main body defining a watertight space, having a removable cap section, and an angled bottom wall for directing the liquid towards the supply line.

7. The system of claim 1, wherein the odor suppressing agent is a liquid, and the reservoir includes a main body defining a watertight space, having a removable cap section that is in direct communication with the dispensing unit.

8. The system of claim 1, wherein the odor suppressing agent is an aerosol gas, and the reservoir consists of an aerosol canister having a first end that is in direct communication with the dispensing unit.

9. The system of claim 1, further comprising a first connector that functions to mount the reservoir to a vehicle.

10. The system of claim 1, further comprising a second connector that functions to mount the dispensing unit to a vehicle dashboard in a removable manner.

11. The system of claim 1, further comprising a third connector that functions to mount the remote activation unit to a vehicle in a removable manner.
